(19) United States
(12) Patent Application Publication
(10) Pub. No.: US 2024/0273728 A1
(43) Pub. Date: Aug. 15, 2024

(54) ANATOMICAL REGION SHAPE PREDICTION

(71) Applicant: KONINKLIJKE PHILIPS N.V., EINDHOVEN (NL)

(72) Inventors: AYUSHI SINHA, BALTIMORE, MD (US); GRZEGORZ ANDRZEJ TOPOREK, CAMBRIDGE, MA (US); LEILI SALEHI, WALTHAM, MA (US); ASHISH SATTYAVRAT PANSE, BURLINGTON, MA (US); RAMON QUIDO ERKAMP, SWAMPSCOTT, MA (US)

(21) Appl. No.: 18/567,074

(22) PCT Filed: Jun. 2, 2022
(86) PCT No.: PCT/EP2022/064991

§ 371 Date: Dec. 5, 2023

Related U.S. Application Data
(60) Provisional application No. 63/208,452, filed on Jun. 8, 2021.

(51) Int. Cl. G06T 7/00; A61B 6/03; A61B 6/50; G06T 7/33; G06T 7/62; G16H 10/60

(52) U.S. Cl. CPC G06T 7/0016; A61B 6/032; A61B 6/504; G06T 7/337; G06T 7/62; G16H 10/60; G06T 2207/10081; G06T 2207/20081; G06T 2207/20084; G06T 2207/30101

(57) ABSTRACT

A computer-implemented method of predicting a shape of an anatomical region includes: receiving (S110) historic volumetric image data (Formula (I)) representing the anatomical region at a historic point in time (t₁): inputting (S120) the received historic volumetric image data (Formula (I)) into a neural network (110): and in response to the inputting (S120), generating (S130). using the neural network (110). predicted sub-sequent volumetric image data (Formula (II)) representing the anatomical region at a subsequent point in time (t₂, tₙ) to the historic point in time (t₁).

### TECHNICAL FIELD

The present disclosure relates to predicting a shape of an anatomical region. A computer-implemented method, a computer program product, and a system, are disclosed.

### BACKGROUND

An aneurism is an unusually-enlarged region of a blood vessel. Aneurisms are caused by weaknesses in the blood vessel wall. Aneurisms can develop in any blood vessel in the body, and most frequently occur in the brain and in the abdominal aorta. Aneurisms require treatment in order to avoid the risk of rupture and consequent internal bleeding and/or haemorrhagic stroke.

The monitoring of aneurisms, and moreover anatomical regions in general, often involves the acquisition of an initial three-dimensional, i.e. volumetric, image of the anatomical region. Subsequently, two-dimensional images of the anatomical region may be acquired over time during follow-up imaging procedures in order to investigate how the anatomical region evolves. The initial volumetric image provides a clinician with detailed information on the anatomical region, and may for example be generated with a computed tomography “CT”, or a magnetic resonance “MR” imaging system. The initial volumetric image may be generated using a contrast agent. CT angiography “CTA”, or MR angiography “MRA” images may for example be generated for this purpose. The two-dimensional images that are acquired during the follow-up imaging procedures may be generated periodically, for example every three months, or at different time intervals. The two-dimensional images are often generated using a projection imaging system such as an X-ray imaging system. A patient's exposure to X-ray radiation may be reduced by generating two-dimensional images instead of volumetric images during the follow-up imaging procedures. The two-dimensional images are often generated using a contrast agent. Digital subtraction angiography “DSA” images, may for example be generated for this purpose. In addition to aneurisms, anatomical regions such as lesions, stenoses, and tumors may also be monitored in this manner.

The ability to accurately evaluate how an anatomical region evolves over time between the acquisition of the initial volumetric image, and the acquisition of the subsequent two-dimensional images at the follow-up imaging procedures, is important since this informs critical decisions such as the follow-up imaging interval, and the need for an interventional procedure.

However, the interpretation of the two-dimensional images is challenging in view of the limited shape information they provide as compared to the initial volumetric image.

Consequently, there is a need for improvements in determining the shape of anatomical regions over time.

### SUMMARY

According to one aspect of the present disclosure, a computer-implemented method of predicting a shape of an anatomical region includes:
receiving historic volumetric image data representing the anatomical region at a historic point in time:
inputting the received historic volumetric image data into a neural network: and in response to the inputting, generating, using the neural network, predicted subsequent volumetric image data representing the anatomical region at a subsequent point in time to the historic point in time: and
wherein the neural network is trained to generate, from volumetric image data representing the anatomical region at a first point in time, predicted volumetric image data representing the anatomical region at a second point in time that is later than the first point in time. Further aspects, features, and advantages of the present disclosure will become apparent from the following description of examples, which is made with reference to the accompanying drawings.

### BRIEF DESCRIPTION OF THE DRAWINGS

FIG. 1 illustrates a DSA image of an aneurism at the top of the basilar artery.

FIG. 2 is a flowchart illustrating a method of predicting a shape of an anatomical region using a neural network, in accordance with some aspects of the present disclosure.

FIG. 3 is a schematic diagram illustrating the inference-time prediction of subsequent volumetric image data [symbol] from historic volumetric image data I₁^{3D} with a neural network 110, in accordance with some aspects of the present disclosure.

FIG. 4 is a flowchart illustrating a method of training a neural network 110 to predict a shape of an anatomical region, in accordance with some aspects of the present disclosure.

FIG. 5 is a schematic diagram illustrating the training of a neural network 110 to predict subsequent volumetric image data [symbol] from historic volumetric image data I₁^{3D}, and wherein the predicted subsequent volumetric image data [symbol] is constrained by subsequent projection image data I₂^{2D} in accordance with some aspects of the present disclosure.

FIG. 6 is a schematic diagram illustrating the training of a neural network 110 to generate predicted volumetric image data [symbol] at a subsequent point in time tₙ, from historic volumetric training image data I₁^{3D} generated at a first point in time t₁, using volumetric training image data Iₙ^{3D} and corresponding two-dimensional training image data Iₙ^{2D} from the subsequent point in time tₙ, and wherein the predicted volumetric image data [symbol] is constrained by the two-dimensional training image data Iₙ^{2D} from the subsequent point in time tₙ, in accordance with some aspects of the present disclosure.

FIG. 7 is a schematic diagram illustrating the inference-time prediction of subsequent volumetric image data [symbol] from historic volumetric image data I₁^{3D}, and wherein the predicted subsequent volumetric image data [symbol] is constrained by subsequent projection image data I₂^{2D} in accordance with some aspects of the present disclosure.

FIG. 8 is a schematic diagram illustrating the inference-time prediction of future volumetric image data [symbol] at a future point in time tₙ₊₁ without constraining the predicted future volumetric image data [symbol] at the future point in time tₙ₊₁ by corresponding projection image data, in accordance with some aspects of the present disclosure.

### DETAILED DESCRIPTION

Examples of the present disclosure are provided with reference to the following description and figures. In this description, for the purposes of explanation, numerous specific details of certain examples are set forth. Reference in the specification to “an example”, “an implementation” or similar language means that a feature, structure, or characteristic described in connection with the example is included in at least that one example. It is to be appreciated that features described in relation to one example may also be used in another example, and that all features are not necessarily duplicated in each example for the sake of brevity. For instance, features described in relation to a computer implemented method, may be implemented in a computer program product, and in a system, in a corresponding manner.

In the following description, reference is made to computer-implemented methods that involve predicting a shape of an anatomical region. Reference is made to an anatomical region in the form of an aneurism. However, it is to be appreciated that the methods may also be used to predict the shape of other anatomical regions in a similar manner. For example, the methods may be used to predict the shapes of lesions, stenoses, and tumors. Moreover, it is to be appreciated that the anatomical region may be located within the vasculature, or in another part of the anatomy.

It is noted that the computer-implemented methods disclosed herein may be provided in the form of a non-transitory computer-readable storage medium including computer-readable instructions stored thereon, which, when executed by at least one processor, cause the at least one processor to perform the method. In other words, the computer-implemented methods may be implemented in a computer program product. The computer program product can be provided by dedicated hardware, or hardware capable of running the software in association with appropriate software. In a similar manner, the computer-implemented methods disclosed herein may be implemented by a system comprising one or more processors that are configured to carry out the methods. When provided by a processor. the functions of the method features can be provided by a single dedicated processor, or by a single shared processor, or by a plurality of individual processors. some of which can be shared. The explicit use of the terms “processor” or “controller” should not be interpreted as exclusively referring to hardware capable of running software. and can implicitly include, but is not limited to. digital signal processor “DSP” hardware. read only memory “ROM” for storing software. random access memory “RAM”. a non-volatile storage device, and the like. Furthermore. examples of the present disclosure can take the form of a computer program product accessible from a computer-usable storage medium, or a computer-readable storage medium. the computer program product providing program code for use by or in connection with a computer or any instruction execution system. For the purposes of this description. a computer-usable storage medium or a computer readable storage medium can be any apparatus that can comprise, store, communicate, propagate, or transport a program for use by or in connection with an instruction execution system. apparatus. or device. The medium can be an electronic. magnetic. optical. electromagnetic. infrared. or a semiconductor system or device or propagation medium. Examples of computer-readable media include semiconductor or solid state memories, magnetic tape. removable computer disks. random access memory “RAM”. read-only memory “ROM”. rigid magnetic disks and optical disks. Current examples of optical disks include compact disk-read only memory “CD-ROM”, compact disk-read/write “CD-R/W”. Blu-Ray™ and DVD.

As mentioned above. the ability to accurately evaluate how an anatomical region evolves over time between the acquisition of an initial volumetric image. and the acquisition of subsequent two-dimensional images at follow-up imaging procedures. is important since this informs critical decisions such as the follow-up imaging interval. and the need for an interventional procedure. However, the interpretation of the subsequent two-dimensional images is challenging in view of the limited shape information they provide as compared to the initial volumetric image.

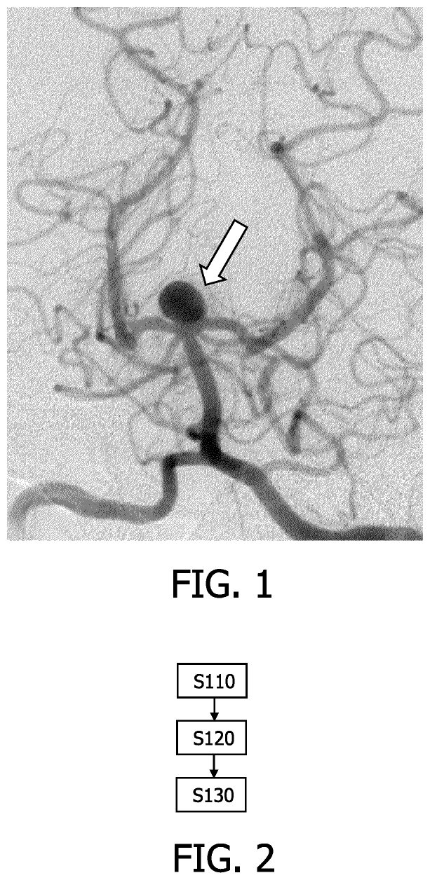

By way of an example. the monitoring of an aneurism over time often involves generating an initial volumetric CT image of the aneurism. and the subsequent generation of two-dimensional DSA projection images during follow-up imaging procedures. DSA imaging employs a contrast agent that highlights the blood flow within the vasculature. FIG. 1 illustrates a DSA image of an aneurism at the top of the basilar artery. The aneurism in Fig. I is indicated by way of the arrow.

As may be appreciated. the two-dimensional projection DSA image in FIG. 1 lacks certain details as compared to a volumetric image. making it difficult to track changes in the aneurism over time. Furthermore. any inconsistencies in the positioning of the patient with respect to the imaging device at each of the follow-up two-dimensional imaging procedures. will result in differing two-dimensional views of the aneurism. These factors create challenges in monitoring the aneurism's evolution over time. As a result. there is a risk that the clinician mis-diagnoses the size of the aneurism. Similarly. there is a risk that the clinician specifics a sub-optimal follow-up interval, or a sub-optimal interventional procedure. or that the aneurism ruptures before a planned intervention.

FIG. 2 is a flowchart illustrating a method of predicting a shape of an anatomical region using a neural network, in accordance with some aspects of the present disclosure. With reference to FIG. 2, a computer-implemented method of predicting a shape of an anatomical region, includes:
receiving S110 historic volumetric image data I₁^{3D} representing the anatomical region at a historic point in time t₁;
inputting S120 the received historic volumetric image data I₁^{3D} into a neural network 110; and
in response to the inputting S120, generating S130, using the neural network 110, predicted subsequent volumetric image data [symbol] , [symbol] representing the anatomical region at a subsequent point in time t₂, tₙ to the historic point in time t₁. The neural network 110 is trained to generate, from volumetric image data representing the anatomical region at a first point in time, predicted volumetric image data representing the anatomical region at a second point in time that is later than the first point in time.

The FIG. 2 method therefore provides a user with the ability to assess how an anatomical region evolves over time. The predicted subsequent volumetric image data [symbol] , [symbol] may be outputted to a display, for example. The user may be provided with the ability to view a depiction of the predicted subsequent volumetric image data from different viewing angles, or to view planar sections through the depiction, and so forth. In the example of the anatomical region being an aneurism, the inputted historic volumetric image data I₁^{3D} can be used to generate predicted 20 subsequent volumetric image data [symbol] , [symbol] at a subsequent point in time that is, for example, three months after the historic volumetric image data I₁^{3D} was acquired. A clinician may use the predicted subsequent volumetric image data to determine whether, and moreover, when, the aneurism is at risk of rupture. Consequently, the FIG. 2 method may allow the clinician to plan an appropriate time for a follow-up imaging procedure, or an interventional procedure on the anatomical region.

The FIG. 2 method is referred-to herein as an inference-time method since predictions, or inferences, are made on the inputted data. Further details of the FIG. 2 method are described with further reference to the Figures below. An associated training method for training the neural network 110 is also described with reference to FIG. 4-FIG. 6.

With reference to the inference-time method of FIG. 2, the historic volumetric image data I₁^{3D} received in the operation S110 may be received via any form of data communication, including wired and wireless communication. By way of some examples, when wired communication is used, the communication may take place via an electrical or optical cable, and when wireless communication is used, the communication may for example be via RF or infrared signals. The 35 historic volumetric image data I₁^{3D} may be received directly from an imaging system, or indirectly, for example via a computer readable storage medium. The historic volumetric image data 130 may for example be received from the internet or the cloud.

The historic volumetric image data I₁^{3D} may be provided by various types of imaging systems, including for example a CT imaging system, an MRI imaging system, an ultrasound imaging system and a positron emission tomography “PET” imaging system. In some examples, a contrast agent may be used to generate the historic volumetric image data I₁^{3D}. Thus, the historic volumetric image data I₁^{3D} that is received in the operation S110 may for example include MRI, CT, MRA, CTA, ultrasound, or PET image data.

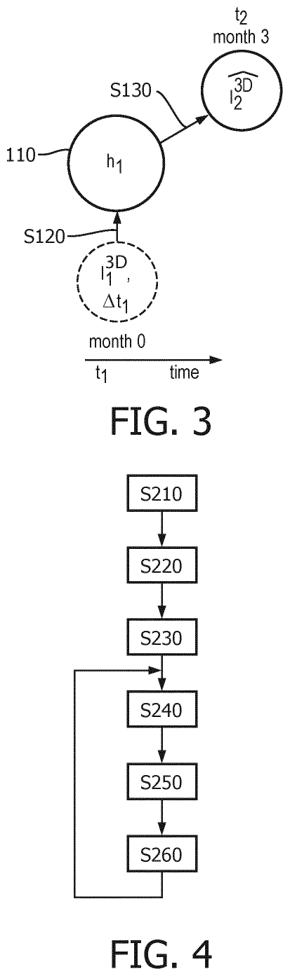

With continued reference to the method of FIG. 2, in the operation S120, the received historic volumetric image data I₁^{3D} is inputted into a trained neural network 110. In this regard, the use of various types of architectures for the neural network 110 is contemplated. In one example, the neural network 110 includes a recurrent neural network “RNN” architecture. A suitable RNN architecture is disclosed in a document by Che, Z. et al. entitled “Recurrent Neural Networks for Multivariate Time Series with Missing Values”. Sci Rep 8, 6085 (2018. https://doi.org/10.1038/s41598-018-24271-9. The RNN may employ long short-term memory “LSTM” units in order to prevent the problem of vanishing gradients during back-propagation. The neural network 110 may alternatively include a different type of architecture, such as a convolutional neural network “CNN” architecture, or a transformer architecture, for example. With continued reference to FIG. 2, in response to the inputting operation S120, predicted subsequent volumetric image data [symbol] , [symbol] is generated in the operation S130. The predicted subsequent volumetric image data [symbol] , [symbol] represents the anatomical region at a subsequent point in time t₂, tₙ to the historic point in time t₁. This is illustrated with reference to FIG. 3, which is a schematic diagram illustrating the inference-time prediction of subsequent volumetric image data [symbol] from historic volumetric image data I₁^{3D} with a neural network 110, in accordance with some aspects of the present disclosure.

The example neural network 110 illustrated in FIG. 3 has an RNN architecture and includes a hidden layer h₁. With reference to FIG. 3, historic volumetric image data I₁^{3D} representing an anatomical region such as an aneurism, or another anatomical region, at a time t₁, i.e. month 0, is inputted into the trained neural network 110 in the operation S120. The predicted subsequent volumetric image data [symbol] that is generated in the operation S130 in response to the inputting represents the anatomical region at a subsequent point in time to the historic point in time t₁, i.e. at t₂ or month 3.

The neural network 110 described with reference to FIG. 2 and FIG. 3 is trained to generate, from volumetric image data representing the anatomical region at a first point in time, predicted volumetric image data representing the anatomical region at a second point in time that is later than the first point in time.

In general. the training of a neural network involves inputting a large training dataset into the neural network. and iteratively adjusting the neural network's parameters until the trained neural network provides an accurate output. Training is often performed using a Graphics Processing Unit “GPU” or a dedicated neural processor such as a Neural Processing Unit “NPU” or a Tensor Processing Unit “TPU”. Training often employs a centralized approach wherein cloud-based or mainframe-based neural processors are used to train a neural network. Following its training with the training dataset. the trained neural network may be deployed to a device for analyzing new input data during inference. The processing requirements during inference are significantly less than those required during training. allowing the neural network to be deployed to a variety of systems such as laptop computers. tablets. mobile phones and so forth. Inference may for example be performed by a Central Processing Unit “CPU”, a GPU, an NPU. a TPU, on a server, or in the cloud.

The process of training the neural network 110 therefore includes adjusting its parameters. The parameters, or more particularly the weights and biases. control the operation of activation functions in the neural network. In supervised learning. the training process automatically adjusts the weights and the biases. such that when presented with the input data. the neural network accurately provides the corresponding expected output data. In order to do this, the value of the loss functions, or errors. are computed based on a difference between predicted output data and the expected output data. The value of the loss function may be computed using functions such as the negative log-likelihood loss. the mean squared error, or the Huber loss. or the cross entropy loss. During training. the value of the loss function is typically minimized. and training is terminated when the value of the loss function satisfies a stopping criterion. Sometimes, training is terminated when the value of the loss function satisfies one or more of multiple criteria.

Various methods are known for solving the loss minimization problem such as gradient descent. Quasi-Newton methods, and so forth. Various algorithms have been developed to implement these methods and their variants including but not limited to Stochastic Gradient Descent “SGD”, batch gradient descent. mini-batch gradient descent. Gauss-Newton. Levenberg Marquardt. Momentum. Adam, Nadam. Adagrad. Adadelta. RMSProp. and Adamax “optimizers” These algorithms compute the derivative of the loss function with respect to the model parameters using the chain rule. This process is called backpropagation since derivatives are computed starting at the last layer or output layer, moving toward the first layer or input layer. These derivatives inform the algorithm how the model parameters must be adjusted in order to minimize the error function. That is. adjustments to model parameters are made starting from the output layer and working backwards in the network until the input layer is reached. In a first training iteration. the initial weights and biases are often randomized. The neural network then predicts the output data. which is likewise, random.

Backpropagation is then used to adjust the weights and the biases. The training process is performed iteratively by making adjustments to the weights and biases in each iteration. Training is terminated when the error, or difference between the predicted output data and the expected output data. is within an acceptable range for the training data, or for some validation data. Subsequently the neural network may be deployed, and the trained neural network makes predictions on new input data using the trained values of its parameters. If the training process was successful, the trained neural network accurately predicts the expected output data from the new input data.

Various examples of methods for training the neural network 110 are described below with reference to FIG. 4-FIG. 6. In these examples, training is performed with a training dataset that includes an initial volumetric image representing an anatomical region, and subsequent two-dimensional images of the anatomical region from subsequent follow-up imaging procedures. A constrained training procedure is employed wherein the neural network 110 uses the initial volumetric image to predict the volumetric shape of the anatomical region at the times of the subsequent two-dimensional images, and the subsequent two-dimensional images are used to constrain the predicted volumetric shape. This method of training the neural network 110 is suited to the availability of existing two-dimensional training data from retrospective imaging procedures.

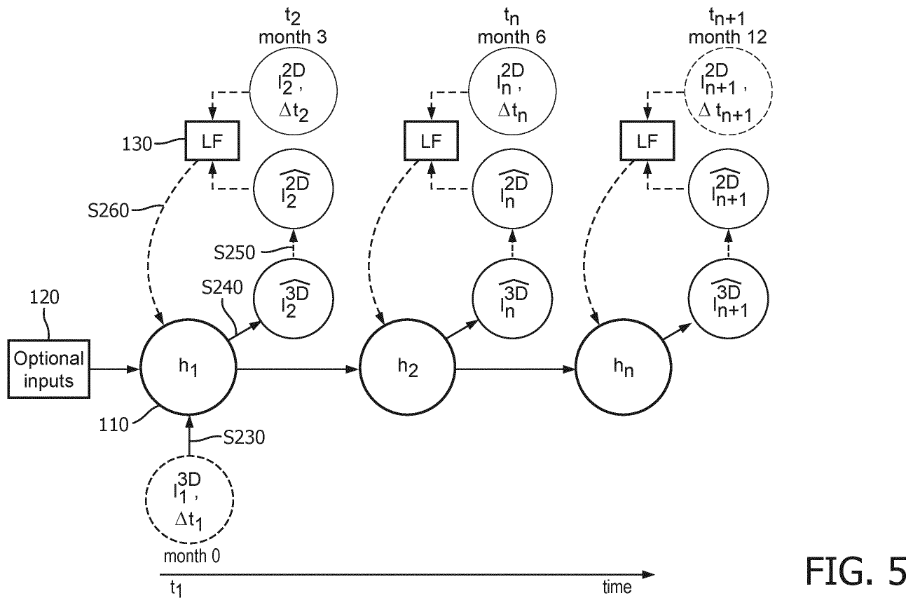

FIG. 4 is a flowchart illustrating a method of training a neural network 110 to predict a shape of an anatomical region, in accordance with some aspects of the present disclosure. FIG. 5 is a schematic diagram illustrating the training of a neural network 110 to predict subsequent volumetric image data [symbol] from historic volumetric image data I₁^{3D}, and wherein the predicted subsequent volumetric image data [symbol] is constrained by subsequent projection image data I₂^{2D} in accordance with some aspects of the present disclosure. In the example RNN illustrated in FIG. 5, there are connections between hidden layers h_{1 . . . n} along the temporal direction that allow the neural network to provide continuity between the predictions over time by incorporating the weights and biases of a previous time step into the predictions made at a subsequent time step. Training involves adjusting the weights and biases of this neural network.

With reference to FIG. 4 and FIG. 5, the neural network 110 is trained to generate, from the volumetric image data representing the anatomical region at the first point in time, the predicted volumetric image data representing the anatomical region at the second point in time, by:
receiving S210 volumetric training image data I₁^{3D} representing the anatomical region at an initial time step t₁;
receiving S220 two-dimensional training image data I₂^{2D}, Iₙ^{2D}, Iₙ₊₁^{2D} representing the anatomical region at a plurality of time steps t₂, tₙ in a sequence after the initial time step t₁;
inputting S230, into the neural network 110, the received volumetric training image data I₁^{3D} for the initial time step t₁; and
for one or more time steps t₂, tₙ, tₙ₊₁ in the sequence after the initial time step t₁;
generating S240, with the neural network 110, predicted volumetric image data [symbol] , [symbol] , [symbol] for the time step t₂, tₙ, tₙ₊₁.
projecting S250 the predicted volumetric image data [symbol] , [symbol] , [symbol] for the time step t₂, tₙ, tₙ₊₁, onto an image plane of the received two-dimensional training image data I₂^{2D}, Iₙ^{2D}, Iₙ₊₁^{2D} for the time step t₂, tₙ, tₙ₊₁; and
adjusting S260 the parameters of the neural network 110 based on a first loss function 130 representing a difference between the projected predicted volumetric image data [symbol] , [symbol] , [symbol] for the time step t₂, tₙ, tₙ₊₁, and the received two-dimensional training image data I₂^{2D}, Iₙ^{2D}, Iₙ₊₁^{2D} for the time step t₂, tₙ, tₙ₊₁.

The volumetric training image data I₁^{3D} that is received in the operation S210 may be provided by any of the imaging systems mentioned above for the historic volumetric image data I₁^{3D}; i.e. it may be provided by a CT imaging system, or an MRI imaging system, or an ultrasound imaging system, or a positron emission tomography “PET” imaging system. Thus, the volumetric training image data I₁^{3D} that is received in the operation S210 may for example include MRI, CT, MRA, CTA, ultrasound, or PET image data.

The volumetric training image data I₁^{3D} that is received in the operation S210 represents the anatomical region at an initial time step t₁. The two-dimensional training image data I₂^{2D}, Iₙ^{2D}, Iₙ₊₁^{2D} that is received in the operation S220 represents the anatomical region at each of a plurality of time steps t₂, tₙ, tₙ₊₁ in a sequence after the initial time step t₁. The use of various types of training image data is contemplated for the two-dimensional training image data I₂^{2D}, Iₙ^{2D}, Iₙ₊₁^{2D}. In some examples, the two-dimensional training image data I₂^{2D}, Iₙ^{2D}, Iₙ₊₁^{2D} is provided by a two-dimensional imaging system, such as for example an X-ray imaging system or a 2D ultrasound imaging system. An X-ray imaging system generates projection data, and therefore the two-dimensional training image data in this former example may be referred-to as projection training image data. In accordance with these examples, the two-dimensional training image data I₂^{2D}, Iₙ^{2D}, Iₙ₊₁^{2D} 221 that is received in the operation S220 may therefore include two-dimensional X-ray image data, contrast-enhanced 2D X-ray image data, 2D DSA image data or 2D ultrasound image data. In some examples however, the two-dimensional training image data I₂^{2D}, Iₙ^{2D} may be generated by projecting volumetric training image data that is generated by a volumetric imaging system such as a CT, or an MRI, or an ultrasound, or a PET, imaging system, onto a plane. Techniques such as ray casting or other known methods may be used to project the volumetric training image data onto a plane. This may be useful in situations where only volumetric training image data is available.

The two-dimensional training image data I₂^{2D}, Iₙ^{2D}, Iₙ₊₁^{2D} may for example be generated periodically, i.e. at regular intervals after the initial time step t₁, for example every three months, or at different intervals after the initial time step t₁: i.e. aperiodically.

The volumetric training image data I₁^{3D}, and the two-dimensional training image data I₂^{2D}, Iₙ^{2D}, Iₙ₊₁^{2D} that are received are the respective operations S210 and S220 may be received via any form of data communication, as mentioned above for the historic volumetric image data I₁^{3D}.

The volumetric training image data I₁^{3D} that is received in the operation S210, and/or the two-dimensional training image data I₂^{2D}, Iₙ^{2D} that is received in the operation S220, may also be annotated. The annotation may be performed manually by an expert user in order to identify the anatomical region, for example the aneurism. Alternatively, the annotation may be performed automatically. In this respect, the use of various automatic image annotation techniques from the image processing field is contemplated, including for example binary segmentation, triangular mesh extracted from binary segmentation for 3D images, and so forth. The use of known image segmentation techniques is contemplated, such as for example: thresholding, template matching, active contour modeling, model-based segmentation, neural networks, e.g., U-Nets, and so forth.

The operations: inputting S230, generating S240, projecting S250 and adjusting S260 that are performed in the above training method are illustrated in FIG. 5 for the time step t₂. The operations: generating S240, projecting S250 and adjusting S260 implement the aforementioned constrained training procedure wherein the neural network uses the initial volumetric image to predict the volumetric shape of the anatomical region at the times of the subsequent two-dimensional images, and the subsequent two-dimensional images are used to constrain the predicted volumetric shape. With reference to FIG. 5, in the operation S240, the volumetric training image data I₁^{3D} at the initial time step t₁ is used to generate predicted volumetric image data [symbol] for the time step t₂. In the operation S250, the predicted volumetric image data [symbol] for the time step t₂ is projected onto an image plane of the two-dimensional training image data I₂^{2D}. In the operation S260, the parameters of the neural network 110 are adjusted based on the value of a first loss function 130. The first loss function 130 represents a difference between the projected predicted volumetric image data [symbol] for the time step t₂, and the received two-dimensional training image data I₂^{2D} for the time step t₂. In so doing, the two-dimensional training image data I₂^{2D} at the time step t₂ is used to constrain the predicted volumetric image data [symbol] .

The operation of constraining of the predicted volumetric shape is therefore implemented by the first loss function 130. Loss functions such as MSE, the L2 loss, or the binary cross entropy loss, and so forth may serve as the first loss function 130. The first loss function may be defined as:

$$LF = BCE ( {I}_{i + 1}^{2 D} , \hat{{I}_{i + 1}^{2 D}} ) \qquad Equation 1$$

The value of the first loss function may be determined by registering the received two-dimensional training image data I₂^{2D} to either the received volumetric image data I₁^{3D} at the initial time step t₁ or the predicted volumetric image data [symbol] for the time step t₂ to determine the plane that the predicted volumetric image data [symbol] or the time step t₂ is projected onto and to generate the projected predicted volumetric image data [symbol] for the time step t₂, and computing a value representing the difference between the projected predicted volumetric image data [symbol] for the time step t₂, and to the received two-dimensional training image data I₂^{2D} for the time step t₂.

In the case where an annotation of an anatomical region is available, the value of the first loss function may be determined by applying a binary mask to the projected predicted volumetric image data [symbol] or the time step t₂, and to the received two-dimensional training image data I₂^{2D} for the time step t₂, and computing a value representing their difference in the annotated region.

After having adjusted the parameters of the neural network 110 in the operation S260, the training method continues by predicting the volumetric image data [symbol] for the next time step in the sequence, i.e. tₙ, and likewise, constraining this prediction with the two-dimensional training image data from the time step tₙ, i.e. Iₙ^{2D}. This is then repeated for all time steps in the sequence, i.e. up to and including time step tₙ₊₁ in FIG. 5.

In so doing, the training method described above with reference to FIG. 4 and FIG. 5 may be used to train the neural network 110 to predict how an anatomical region, such as an aneurism, evolves over time. When trained, the neural network 110 illustrated in FIG. 5 can then predict the future shape of an anatomical region such as an aneurism from an inputted historic volumetric image in the absence of any two-dimensional image. The training method can therefore be used to provide the neural network 110 illustrated in FIG. 3.

Whilst the training method was described above for an anatomical region in a single subject, the training may be performed for the anatomical region in multiple subjects. The training image data may for example be provided for more than a hundred subjects across different age groups, genders, body mass index, abnormalities in the anatomical region, and so forth. Thus, in one example, the received volumetric training image data I₁^{3D} represents the anatomical region at an initial time step t₁ in a plurality of different subjects: and the received two-dimensional training image data I₂^{2D}, Iₙ^{2D} comprises a plurality of sequences, each sequence representing the anatomical region in a corresponding subject at a plurality of time steps t₂, tₙ in a sequence after the initial time step t₁ for the corresponding subject: and the inputting S230, the generating S240, the projecting S250, and the adjusting S260, are performed with the received volumetric training image data I₁^{3D} and the received two-dimensional training image data I₂^{2D}, Iₙ^{2D} for each subject.

As mentioned above, in the projecting operation S250, the image plane of the received two-dimensional training image data I₂^{2D}, Iₙ^{2D} for the time step t₂, tₙ may be determined by i) registering the received two-dimensional training image data I₂^{2D}, Iₙ^{2D} for the time step t₂, tₙ to the received volumetric training image data I₂^{2D}, Iₙ^{2D} for the initial time step t₁, or by ii) registering the received two-dimensional training image data I₂^{2D}, Iₙ^{2D} for the time step t₂, tₙ to the predicted volumetric training image data [symbol] , [symbol] for the time step t₂, tₙ. Various known image registration techniques may be used for this purpose.

As mentioned above, anatomical regions are often monitored over time by generating an initial volumetric image, and then generating projection images at subsequent follow-up imaging procedures. This provides a certain amount of training image data that may, as described above, be used to train the neural network 110. In some cases however, additional volumetric image data may also be available from such monitoring procedures, presenting the opportunity for volumetric image data to be used in combination with the two-dimensional training image data I₂^{2D}, Iₙ^{2D} to train the neural network 110. The use of the additional volumetric image data may provide improved, or faster, training of the neural network 110. Thus, in one example, the above-described training method is adapted, and the neural network 110 is trained to predict the volumetric image data representing the anatomical region at the second point in time, by further:
receiving volumetric training image data I₂^{3D}, Iₙ^{3D} corresponding to the two-dimensional training image data I₂^{2D}, Iₙ^{2D} at one or more of the time steps t₂, tₙ in the sequence after the initial time step t₁; and
wherein the adjusting S260 is based further on a second loss function 140 representing a difference between the predicted volumetric image data [symbol] , [symbol] for the time step t₂, tₙ, and the received volumetric training image data I₂^{3D}, Iₙ^{3D} for the time step t₂, tₙ.

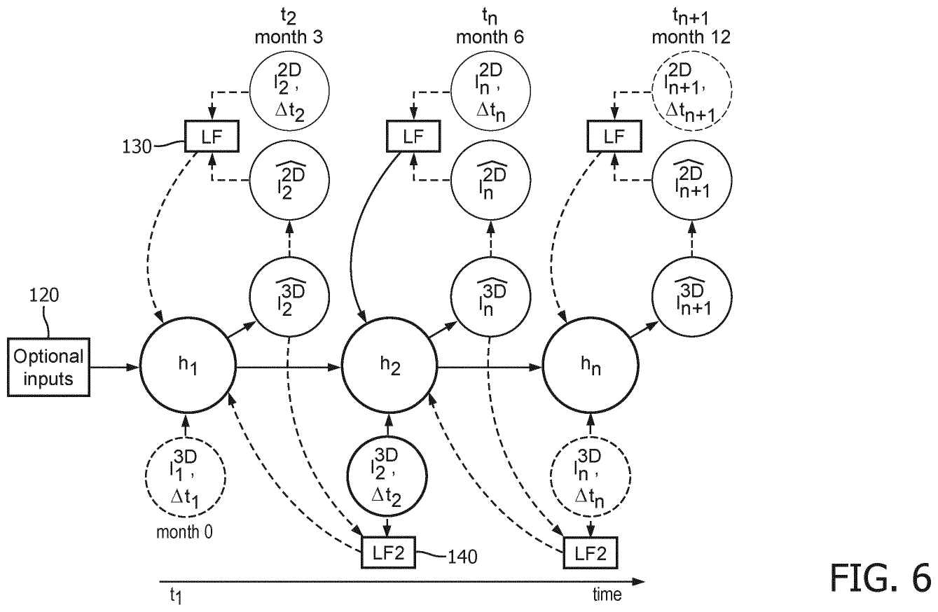

This example is described with reference to FIG. 6, which is a schematic diagram illustrating the training of a neural network 110 to generate predicted volumetric image data [symbol] at a subsequent point in time tₙ, from historic volumetric training image data I₁^{3D} generated at a first point in time t₁, using volumetric training image data Iₙ^{3D} and corresponding two-dimensional training image data Iₙ^{2D} from the subsequent point in time tₙ, and wherein the predicted volumetric image data [symbol] is constrained by the two-dimensional training image data Iₙ^{2D} from the subsequent point in time tₙ, in accordance with some aspects of the present disclosure. The training method illustrated in FIG. 6 differs from the training method illustrated in FIG. 5 in that volumetric training image data I₂^{3D}, Iₙ^{3D} is also used to train the neural network 110 in FIG. 6, and FIG. 6 also includes a second loss function 140 that is used to determine a difference between the predicted volumetric image data [symbol] , [symbol] and the received volumetric training image data I₂^{3D}, Iₙ^{3D}.

The volumetric training image data I₂^{3D}, Iₙ^{3D} that is used in the FIG. 6 neural network 110 may be provided by any of the imaging systems mentioned above that is used to generate the volumetric training image data I₁^{3D} that is inputted in the operation S230. The volumetric training image data I₂^{3D}, Iₙ^{3D} corresponds to the two-dimensional training image data I₂^{2D}, Iₙ^{2D} in the sense that they both represent the same anatomical region, and they are generated simultaneously, or within a short time interval of one another. For example, the volumetric training image data I₂^{3D}, Iₙ^{3D} and the two-dimensional training image data I₂^{2D}, Iₙ^{2D} may be generated within a few hours of one another, or on the same day as one another. Alternatively, if only volumetric training image data I₂^{3D}, Iₙ^{3D} is acquired, corresponding two-dimensional training image data I₂^{2D}, Iₙ^{2D} may be generated by projecting the volumetric image data onto a plane using ray casting or other established methods to generate two-dimensional images from volumetric data.

The second loss function 140 described with reference to FIG. 6 may be provided by any of the loss functions mentioned above in relation to the first loss function 130. The value of the second loss function may, likewise, be determined by registering the predicted volumetric image data [symbol] to the volumetric training image data I₂^{3D}, and computing a value representing their difference. As mentioned above, in the case where an annotation of an anatomical region is available, the value of the second loss function may be determined by applying a binary mask to the predicted volumetric image data [symbol] for the time step t₂, and to the volumetric training image data I₂^{3D} for the time step t₂. registering the predicted volumetric image data [symbol] to the volumetric training image data I₂^{3D}, and computing a value representing their difference in the annotated region.

The predictions of the neural network 110 described above may in general be improved by training the neural network to predict the volumetric image data [symbol] , [symbol] , [symbol] based further on the time difference between when the historic volumetric image data I₁^{3D} was acquired, and the time of the prediction, i.e. the time difference between the historic point in time t₁, and the time t₂, or tₙ, or tₙ₊. This time difference is illustrated in the Figures by the symbols Dt₁, Dt₂, and Dtₙ, respectively. In the illustrated example, Dt₁ may be zero. Basing the predictions of the neural network 110 on this time difference allows the neural network 110 to learn the association between a length of the time difference, and changes in the anatomical region. Thus, in one example, the neural network 110 is trained to generate the predicted volumetric image data representing the anatomical region at the second point in time, based further on a time difference between the first point in time and the second point in time, and the inference-time method also includes:
inputting, into the neural network 110, a time difference Dt₁ between the historic point in time t₁ and the subsequent point in time t₂, tₙ, and generating S130, using the neural network 110, the predicted subsequent volumetric image data [symbol] , [symbol] based further on the time difference Dt₁.

In practice, the time difference that is used may depend on factors such as type of the anatomical region, the rate at which it is expected to evolve, and the severity of its condition. In the example of the anatomical region being an aneurism, follow-up imaging procedures are often performed at three-monthly intervals, and so the time difference may for example be set to three months. In general however, the time interval may be set to any value, and the time interval may be periodic, or aperiodic.

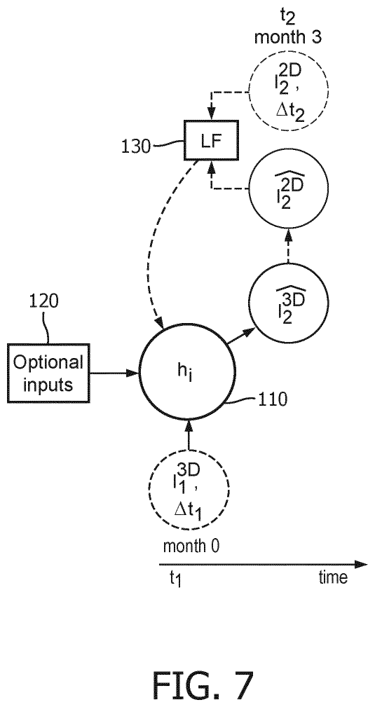

As mentioned above, in some cases, anatomical regions are monitored by acquiring an initial volumetric image, i.e. the historic volumetric image data I₁^{3D}, and subsequently acquiring two-dimensional image data, or more specifically, projection image data of the anatomical region over time. The projection image data may be generated by an X-ray imaging system. In accordance with one example, projection image data is used at inference-time to constrain the predictions of the volumetric image data [symbol] , [symbol] . This constraining is performed in a similar manner to the constrained training operation that was described above. Constraining the predictions of the neural network 110 at inference-time in this manner may provide a more accurate prediction of the volumetric image data. FIG. 7 is a schematic diagram illustrating the inference-time prediction of subsequent volumetric image data [symbol] from historic volumetric image data I₁^{3D}, and wherein the predicted subsequent volumetric image data [symbol] is constrained by subsequent projection image data I₂^{2D} in accordance with some aspects of the present disclosure. In addition to the operations described in relation to FIG. 3, in the inference-time method illustrated in FIG. 7, subsequent projection image data I₂^{2D} from the subsequent point in time t₂, is used to constrain the predicted volumetric image data [symbol] at the subsequent point in time t₂, by means of a first loss function 130.

In the FIG. 7 method, the neural network 110 is trained to generate the predicted volumetric image data [symbol] [symbol] representing the anatomical region at the second point in time such that the predicted volumetric image data is constrained by projection image data representing the anatomical region at the second point in time. In addition to the operations described above with reference to FIG. 3, the inference-time method also includes:
receiving subsequent projection image data I₂^{2D}, Iₙ^{2D} representing the anatomical region at the subsequent point in time t₂, tₙ: and
wherein the generating S130 is performed such that the predicted subsequent volumetric image data /20,130 representing the anatomical region at the subsequent point in time t₂, tₙ is constrained by the subsequent projection image data I₂^{2D}, Iₙ^{2D}.

In the FIG. 7 method, the predicted subsequent volumetric image data [symbol] , [symbol] is constrained by the first loss function 130. The first loss function 130 operates in the same manner as described above for the first loss function 130 in FIG. 5 that was used during training, with the exception that the inputted projection image data I₂^{2D} in FIG. 7 is not training data as in FIG. 5, and is instead data that is acquired at inference time.

With reference to FIG. 7, the subsequent projection image data I₂^{2D}, Iₙ^{2D} may be provided by various types of projection imaging systems, including the aforementioned X-ray imaging system. A similar first loss function 130 to that described with reference to FIG. 5, may also be used at inference-time in order to constrain the predicted subsequent volumetric image data [symbol] , [symbol] .

Additional input data may also be inputted into the neural network 110 during training, and likewise during inference, and used by the neural network to predict the subsequent volumetric image data [symbol] , [symbol] . For example, the time difference Dt₁ between the historic point in time t₁ and the subsequent point in time t₂, tₙ may be inputted into the neural network, and the neural network 110, may generate the predicted subsequent volumetric image data [symbol] , [symbol] based further on the time difference Dt₁. In another example, the neural network 110 is further trained to predict the volumetric image data based on patient data 120, and the inference-time method further includes:
inputting patient data 120 into the neural network 110: and
generating the predicted subsequent volumetric image data [symbol] , [symbol] based on the patient data 120.

Examples of patient data 120 include patient gender, patient age, a patient's blood pressure, a patient's weight, a patient's genomic data (including e.g. genomic data representing endothelial function), a patient's heart health status, a patient's treatment history, a patient's smoking history, a patient's family health history, a type of the aneurism, and so forth. Using the patient data in this manner may improve the predictions of the neural network 110 since this information affects changes in anatomical regions, for instance, the rate of growth of aneurisms.

The inference-time method may additionally include an operation of computing a measurement of the anatomical region represented in the predicted subsequent volumetric image data [symbol] , [symbol] ; and/or an operation of generating one or more clinical recommendations based on the predicted subsequent volumetric image data [symbol] , [symbol] .

Measurements of the anatomical region such as its volume, its change in volume since the previous imaging procedure, its rate of change in volume, its diameter, the aneurism neck diameter, in the example of the anatomical region being an aneurism, and so forth may, be computed by post-processing the volumetric image data [symbol] , [symbol] that is predicted by the neural network 110. The clinical recommendations may likewise be computed by post-processing the volumetric image data [symbol] , [symbol] , or alternatively outputted by the neural network. Example clinical recommendations include the suggested time of a future follow-up imaging procedure, the suggested type of follow-up imaging procedure, and the need for a clinical intervention such as an embolization procedure or a flow-diverting stent in the example of the anatomical region being an aneurism. In the example of the anatomical region being an aneurism, the risk of rupture at a particular point in time may also be calculated and outputted. These recommendations may be based on the predicted measurements, for example based on the predicted volume, or the predicted rate of growth of the anatomical region. For example, the recommendation may be contingent on the predicted volume or the predicted rate of growth of the anatomical region exceeding a threshold value.

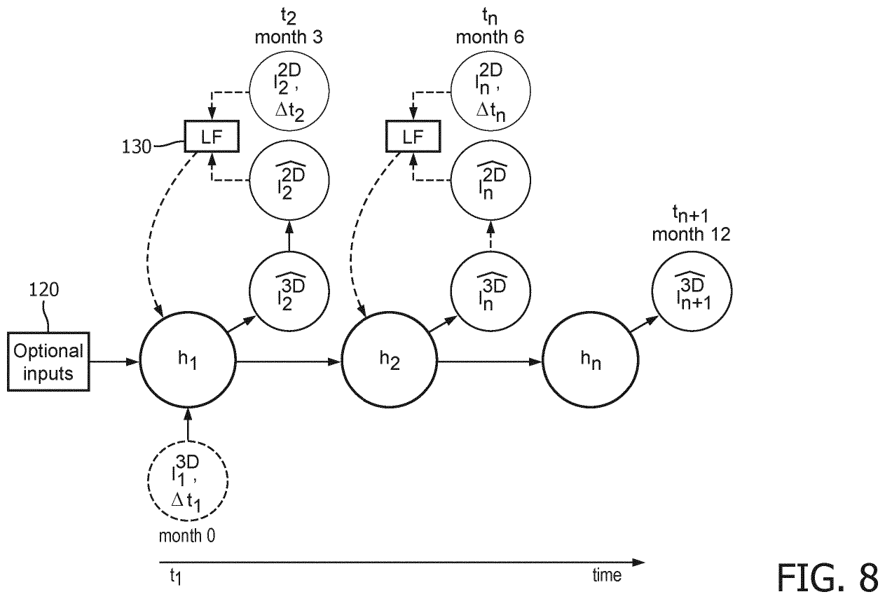

In some cases, during the monitoring of an anatomical region, historic volumetric image data I₁^{3D} is available for an anatomical region in a patient, together with one or more projection images of the anatomical region that have been acquired at subsequent follow-up imaging procedures. A physician may be interested in the subsequent evolution of the anatomical region at a future point in time. The physician may for example want to predict the volumetric image data in order to propose the time of the next follow-up imaging procedure. In this situation, no projection image data is yet available for the future point in time. However, in one example, the trained neural network 110 may be used to make a constrained prediction of the volumetric image data [symbol] , [symbol] for one or more time intervals, these constrained predictions being constrained by the projection image data that is available, and to make an unconstrained prediction of the volumetric image data for the future point in time of the proposed follow-up imaging procedure. The unconstrained prediction is possible because, as described above, during inference, it is not essential to the trained neural network to constrain its predictions with the projection image data. The projection image data simply improves the predictions of the neural network. The unconstrained prediction can be made by using the trained neural network, which may indeed be a neural network that is trained to make constrained predictions, and making the unconstrained prediction for the future point in time without the use of any projection data. In this regard, FIG. 8 is a schematic diagram illustrating the inference-time prediction of future volumetric image data [symbol] at a future point in time tₙ₊₁ without constraining the predicted future volumetric image data [symbol] at the future point in time tₙ₊₁ by corresponding projection image data, in accordance with some aspects of the present disclosure.

With reference to FIG. 8, historic volumetric image data I₁^{3D} is available for an anatomical region at time t₁. Projection images of the anatomical region, i.e. projection image data I₂^{2D}, Iₙ^{2D}, are available for the subsequent points in time t₂ and tₙ, and are used to make respective constrained predictions of the volumetric image data [symbol] and [symbol] at times t₂ and tₙ. The clinician is however interested in how the anatomical region might appear at a future point in time tₙ₊₁. Since no projection image data is available to constrain the prediction at time tₙ₊₁, an unconstrained prediction is made for time tₙ₊₁. In this example, at inference-time, the trained neural network 110 generates predicted future volumetric image data [symbol] representing the anatomical region at a future point in time tₙ₊₁ that is later than the subsequent point in time tₙ, without constraining the predicted future volumetric image data [symbol] at the future point in time tₙ.1 by corresponding projection image data.

In the inference-time method, the constrained predictions of the volumetric image data [symbol] and [symbol] can be made using the projection image data I₂^{2D}, Iₙ^{2D} by projecting the volumetric image data onto the image plane of the projection image data I₂^{2D}, Iₙ^{2D}. Thus, in the inference-time method, the operation of generating S130, using the neural network 110, predicted subsequent volumetric image data [symbol] , [symbol] representing the anatomical region at the subsequent point in time t₂, tₙ that is constrained by the subsequent projection image data I₂^{2D}, may include:
projecting the predicted subsequent volumetric image data [symbol] , [symbol] representing the anatomical region at the subsequent point in time t₂, tₙ, onto an image plane of the received subsequent projection image data I₂^{2D}, Iₙ^{2D}, and generating, using the neural network 110, the predicted subsequent volumetric image data [symbol] , [symbol] representing the anatomical region at the subsequent point in time t₂, tₙ based on a difference between the projected predicted subsequent volumetric image data [symbol] , [symbol] representing the anatomical region at the subsequent point in time t₂. tₙ, and the subsequent projection image data I₂^{2D}, Iₙ^{2D}.

The difference between the projected predicted subsequent volumetric image data [symbol] , [symbol] representing the anatomical region at the subsequent point in time t₂, tₙ, and the subsequent projection image data I₂^{2D}, Iₙ^{2D} may be computed using a loss function, as indicated by first loss function 130 in FIG. 7. Various loss functions may be used to compute the first loss function 130 that is used in the adjusting operation S260. For example, MSE, the L2 loss, or the binary cross entropy loss, etc. may be used.

The image plane of the received subsequent projection image data I₂^{2D}, Iₙ^{2D} may be determined by i) registering the received subsequent projection image data I₂^{2D}, Iₙ^{2D} to the received historic volumetric image data I₁^{3D}, or by ii) registering the received subsequent projection image data I₂^{2D}, Iₙ^{2D} to the predicted subsequent volumetric image data [symbol] , [symbol] .

In some examples, at inference-time, the anatomical region may also be segmented in the historic volumetric image data I₁^{3D} and/or in the subsequent projection image data I₂^{2D}, Iₙ^{2D}, prior to, respectively, inputting S120 the received historic volumetric image data I₁^{3D} into the neural network 110 and/or using the received subsequent projection image data I₂^{2D}, Iₙ^{2D} to constrain the predicted subsequent volumetric image data [symbol] , [symbol] . The segmentation may improve the predictions made by the neural network. The use of similar segmentations to those used in training the neural network is contemplated, including: thresholding, template matching, active contour modeling, model-based segmentation, neural networks e.g., U-Nets, and so forth.

In some examples, the inference-time method may also include the operation of generating a confidence estimate of the predicted subsequent volumetric image data [symbol] , [symbol] A confidence estimate may be computed based on the quality of the inputted projection image data and/or the quality of the inputted volumetric image data, such as the amount of blurriness in the image caused by movement during image acquisition, the amount of contrast flowing through the aneurysm, and so forth. The confidence estimate may be outputted as a numerical value, for example. In examples wherein the predicted subsequent volumetric image data [symbol] , [symbol] is constrained by subsequent projection image data I₂^{2D}, the confidence estimate may be based on the difference between a projection of the predicted subsequent volumetric image data [symbol] , [symbol] for the time step t₂, tₙ, onto an image plane of the received two-dimensional training image data I₂^{2D}, Iₙ^{2D} for the time step t₂, tₙ, and the subsequent projection image data I₂^{2D}, Iₙ^{2D} for the time step t₂, tₙ. A value of the confidence estimate may be computed from the value of the loss function 130 described in relation to FIG. 5, FIG. 7 and FIG. 8, or by computing another metric such as the intersection over union “IoU”, or the dice coefficient, and so forth.

At inference time, or during training, the above methods may be accelerated by limiting the predicted volumetric image data [symbol] , [symbol] to particular regions. Thus, in some examples, the inference-time method may also include:
i) receiving input indicative of a bounding volume defining an extent of the anatomical region in the received historic volumetric image data I₁^{3D}; and
   wherein the generating S130, using the neural network 110, the predicted subsequent volumetric image data [symbol] , [symbol] , is constrained by generating the predicted subsequent volumetric image data [symbol] , [symbol] only within the bounding volume:
and/or
ii) receiving input indicative of a bounding area defining an extent of the anatomical region in the received subsequent projection image data I₂^{2D}; and
   wherein the generating S130, using the neural network 110, the predicted subsequent volumetric image data [symbol] , [symbol] , is constrained by generating the predicted subsequent volumetric image data [symbol] , [symbol] for a volume corresponding to the bounding area in the received subsequent projection image data I₂^{2D}.

Without prejudice to the generality of the above, in one group of Examples, the neural network 110 is trained to generate the predicted volumetric image data representing the anatomical region at the second point in time such that the predicted volumetric image data is constrained by projection image data representing the anatomical region at the second point in time. These Examples are enumerated below:

Example 1. A computer-implemented method of predicting a shape of an anatomical region, the method comprising;
receiving S110 historic volumetric image data I₁^{3D} representing the anatomical region at a historic point in time t¹;
receiving subsequent projection image data I₂^{2D}, Iₙ^{2D} representing the anatomical region at the subsequent point in time t₂, tₙ;
inputting S120 the received historic volumetric image data I₁^{3D} into a neural network 110; and
in response to the inputting S120, generating S130, using the neural network 110, predicted subsequent volumetric image data [symbol] , [symbol] representing the anatomical region at a subsequent point in time t₂, tₙ to the historic point in time t₁ that is constrained by the subsequent projection image data I₂^{2D}, Iₙ^{2D} and
wherein the neural network 110 is trained to generate, from volumetric image data representing the anatomical region at a first point in time, predicted volumetric image data representing the anatomical region at a second point in time that is later than the first point in time such that the predicted volumetric image data is constrained by projection image data representing the anatomical region at the second point in time.

Example 2. The computer-implemented method according to Example 1, wherein the method further comprises:
inputting, into the neural network 110, a time difference Dt₁ between the historic point in time t₁ and the subsequent point in time t₂, tₙ, and generating S130, using the neural network 110, the predicted subsequent volumetric image data [symbol] , [symbol] based further on the time difference Dt₁; and
wherein the neural network 110 is trained to generate the predicted volumetric image data representing the anatomical region at the second point in time, based further on a time difference between the first point in time and the second point in time.

Example 3. The computer-implemented method according to Example 1 or Example 2,further comprising:
generating, using the neural network 110, predicted future volumetric image data [symbol] representing the anatomical region at a future point in time tₙ₊₁ that is later than the subsequent point in time tₙ, without constraining the predicted future volumetric image data [symbol] at the future point in time tₙ+1 by corresponding projection image data.

Example 4. The computer-implemented method according to any previous Example, further comprising segmenting the anatomical region in the received historic volumetric image data I₁^{3D} and/or in the received subsequent projection image data I₂^{2D}, Iₙ^{2D}, prior to, respectively, inputting S120 the received historic volumetric image data I₁^{3D} into the neural network 110 and/or using the received subsequent projection image data I₂^{2D}, Iₙ^{2D} to constrain the predicted subsequent volumetric image data [symbol] , [symbol] .

Example 5. The computer-implemented method according to any previous Example, wherein the generating S130, using the neural network 110, predicted subsequent volumetric image data [symbol] , [symbol] representing the anatomical region at the subsequent point in time t₂, tₙ that is constrained by the subsequent projection image data I₂^{2D}, comprises:
projecting the predicted subsequent volumetric image data [symbol] , [symbol] representing the anatomical region at the subsequent point in time t₂, tₙ, onto an image plane of the received subsequent projection image data I₂^{2D}, Iₙ^{2D}, and generating, using the neural network 110, the predicted subsequent volumetric image data [symbol] , [symbol] representing the anatomical region at the subsequent point in time t₂, tₙ based on a difference between the projected predicted subsequent volumetric image data [symbol] , [symbol] representing the anatomical region at the subsequent point in time t₂, tₙ, and the subsequent projection image data I₂^{2D}, Iₙ^{2D}.

Example 6. The computer-implemented method according to Example 5, wherein the image plane of the received subsequent projection image data I₂^{2D}, Iₙ^{2D} is determined by i) registering the received subsequent projection image data I₂^{2D}, Iₙ^{2D} to the received historic volumetric image data I₁^{3D}, or by ii) registering the received subsequent projection image data I₂^{2D}, Iₙ^{2D} to the predicted subsequent volumetric image data [symbol] , [symbol] .

Example 7. The computer-implemented method according to any previous Example, further comprising:
inputting patient data 120 into the neural network 110; and
generating the predicted subsequent volumetric image data [symbol] , [symbol] based on the patient data 120; and
wherein the neural network 110 is further trained to predict the volumetric image data based on patient data 120.

Example 8. The computer-implemented method according to any previous Example, further comprising:
computing a measurement of the anatomical region represented in the predicted subsequent volumetric image data [symbol] , [symbol] ; and/or
generating one or more clinical recommendations based on the predicted subsequent volumetric image data [symbol] , [symbol] .

Example 9. The computer-implemented method according to any previous Example, further comprising:
i) receiving input indicative of a bounding volume defining an extent of the anatomical region in the received historic volumetric image data I₁^{3D}; and
   wherein the generating S130, using the neural network 110, the predicted subsequent volumetric image data [symbol] , [symbol] , is constrained by generating the predicted subsequent volumetric image data [symbol] , [symbol] only within the bounding volume:
and/or
receiving input indicative of a bounding area defining an extent of the anatomical region in the received subsequent projection image data I₂^{2D}; and
   wherein the generating S130, using the neural network 110, the predicted subsequent volumetric image data [symbol] , [symbol] , is constrained by generating the predicted subsequent volumetric image data [symbol] , [symbol] for a volume corresponding to the bounding area in the received subsequent projection image data I₂^{2D}.

Example 10. The computer-implemented method according to Example 1, wherein the neural network 110 is trained to generate, from the volumetric image data representing the anatomical region at the first point in time, the predicted volumetric image data representing the anatomical region at the second point in time such that the predicted volumetric image data is constrained by the projection image data representing the anatomical region at the second point in time, by:
receiving S210 volumetric training image data I₁^{3D} representing the anatomical region at an initial time step t₁;
receiving S220 two-dimensional training image data I₂^{2D}, Iₙ^{2D} representing the anatomical region at a plurality of time steps t₂, tₙ in a sequence after the initial time step t₁;
inputting S230, into the neural network 110, the received volumetric training image data I₁^{3D} for the initial time step t₁; and
for one or more time steps t₂, tₙ in the sequence after the initial time step t₁;
generating S240, with the neural network 110, predicted volumetric image data [symbol] , [symbol] for the time step t₂, tₙ;
projecting S250 the predicted volumetric image data [symbol] , [symbol] for the time step t₂, tₙ, onto an image plane of the received two-dimensional training image data I₂^{2D}, Iₙ^{2D} for the time step t₂, tₙ; and
adjusting S260 the parameters of the neural network 110 based on a first loss function 130 representing a difference between the projected predicted volumetric image data [symbol] , [symbol] for the time step t₂, tₙ, and the received two-dimensional training image data I₂^{2D}, Iₙ^{2D} for the time step t₂, tₙ.

Example 11. The computer-implemented method according to Example 10, wherein the neural network 110 is trained to predict the volumetric image data representing the anatomical region at the second point in time, by further:
receiving volumetric training image data I₂^{3D}, Iₙ^{3D} corresponding to the two-dimensional training image data I₂^{2D}, Iₙ^{2D} at one or more of the time steps t₂, tₙ in the sequence after the initial time step t₁; and
wherein the adjusting S260 is based further on a second loss function 140 representing a difference between the predicted volumetric image data [symbol] , [symbol] for the time step t₂, tₙ, and the received volumetric training image data I₂^{3D}, Iₙ^{3D} for the time step t₂, tₙ.

Example 12. The computer-implemented method according to Example 10 or Example 11, wherein the image plane of the received two-dimensional training image data I₂^{2D}, Iₙ^{2D} for the time step t₂, tₙ is determined by i) registering the received two-dimensional training image data I₂^{2D}, Iₙ^{2D} for the time step t₂, tₙ to the received volumetric training image data I₁^{3D} for the initial time step t₁, or by ii) registering the received two-dimensional training image data I₂^{2D}, Iₙ^{2D} for the time step t₂, tₙ to the predicted volumetric training image data [symbol] , [symbol] for the time step t₂, tₙ.

Example 13. The computer-implemented method according to Example 10: wherein the received volumetric training image data 130 represents the anatomical region at an initial time step t₁ in a plurality of different subjects:

wherein the received two-dimensional training image data 120, 12D comprises a plurality of sequences, each sequence representing the anatomical region in a corresponding subject at a plurality of time steps t₂, tₙ in a sequence after the initial time step t₁ for the corresponding subject: and
wherein the inputting S230, the generating S240, the projecting S250, and the adjusting S260, are performed with the received volumetric training image data I₁^{3D} and the received two-dimensional training image data I₂^{2D}, Iₙ^{2D} for each subject.

Example 14. A computer program product comprising instructions which when executed by one or more processors, cause the one or more processors to carry out the method according to any one of Examples 1-13.

Example 15. A system for predicting a shape of an anatomical region, the system comprising one or more processors configured to:
receive S110 historic volumetric image data I₁^{3D} representing the anatomical region at a historic point in time t₁;
receive subsequent projection image data I₂^{2D}, Iₙ^{2D} representing the anatomical region at the subsequent point in time t₂, tₙ;
input S120 the received historic volumetric image data I₁^{3D} into a neural network 110; and
in response to the inputting S120, generate S130, using the neural network 110, predicted subsequent volumetric image data [symbol] , [symbol] representing the anatomical region at a subsequent point in time t₂, tₙ to the historic point in time t₁ that is constrained by the subsequent projection image data I₂^{2D}, Iₙ^{2D}; and
wherein the neural network 110 is trained to generate, from volumetric image data representing the anatomical region at a first point in time, predicted volumetric image data representing the anatomical region at a second point in time that is later than the first point in time such that the predicted volumetric image data is constrained by projection image data representing the anatomical region at the second point in time.

The above examples are to be understood as illustrative of the present disclosure, and not restrictive. Further examples are also contemplated. For instance, the examples described in relation to computer-implemented methods, may also be provided by a computer program product, or by a computer-readable storage medium, or by a system, in a corresponding manner. It is to be understood that a feature described in relation to any one example may be used alone, or in combination with other described features, and may be used in combination with one or more features of another of the examples, or a combination of other examples. Furthermore, equivalents and modifications not described above may also be employed without departing from the scope of the invention, which is defined in the accompanying claims. In the claims, the word “comprising” does not exclude other elements or operations, and the indefinite article “a” or “an” does not exclude a plurality. The mere fact that certain features are recited in mutually different dependent claims does not indicate that a combination of these features cannot be used to advantage. Any reference signs in the claims should not be construed as limiting their scope.

1. A computer-implemented method of predicting a shape of an anatomical region, the method comprising:
receiving historic volumetric image data representing the anatomical region at a historic point in time;
receiving subsequent projection image data representing the anatomical region at a subsequent point in time that is subsequent to the historical point in time; and
predicting subsequent volumetric image data representing the anatomical region at the subsequent point in time based on the historic volumetric image data and the subsequent projection image data, wherein the prediction of the subsequent volumetric image data is constrained by the subsequent projection image data.

2. The computer-implemented method according to claim 16, further comprising:
inputting, into the neural network, a time difference between the historic point in time and the subsequent point in time, and generating (S130), using the neural network, the predicted subsequent volumetric image data based further on the time difference; and
wherein the neural network is trained to predict second volumetric image data based further on a time difference between the first point in time and the second point in time.

3. The computer-implemented method according to claim 16, wherein the region of interest is an aneurism, the historic volumetric image data is an initial volumetric CT image of the aneurism, and the subsequent projection image data is a two-dimensional DSA projection image being acquired during an imaging procedure.

4. The computer-implemented method according to claim 3, further comprising:
predicting, using the neural network, future volumetric image data representing the anatomical region at a future point in time that is later than the subsequent point in time, without constraining the predicted future volumetric image data at the future point in time by corresponding projection image data.

5. The computer-implemented method according to claim 1, further comprising:
segmenting the anatomical region in at least one of the received historic volumetric image data or the received subsequent projection image data, prior to, respectively, at least one of inputting the received historic volumetric image data into the neural network and/or using the received subsequent projection image data to constrain the predicted subsequent volumetric image data.

6. The computer-implemented method according to claim 3, wherein the predicting, using the neural network, of the subsequent volumetric image data representing the anatomical region at the subsequent point in time that is constrained by the subsequent projection image data, comprises:
projecting the predicted subsequent volumetric image data representing the anatomical region at the subsequent point in time, onto an image plane of the received subsequent projection image data, and generating, using the neural network, the predicted subsequent volumetric image data representing the anatomical region at the subsequent point in time based on a difference between the projected predicted subsequent volumetric image data representing the anatomical region at the subsequent point in time, and the subsequent projection image data.

7. The computer-implemented method according to claim 6, wherein the image plane of the received subsequent projection image data is determined by i) registering the received subsequent projection image data to the received historic volumetric image data, or by ii) registering the received subsequent projection image data to the predicted subsequent volumetric image data.

8. The computer-implemented method according to claim 16, further comprising:
inputting patient data into the neural network ; and
generating the predicted subsequent volumetric image data based on the patient data; and
wherein the neural network is further trained to predict volumetric image data based on patient data.

9. The computer-implemented method according to claim 1, further comprising at least one of:
computing a measurement of the anatomical region represented in the predicted subsequent volumetric image data or
generating one or more clinical recommendations based on the predicted subsequent volumetric image data.

10. The computer-implemented method according to claim 16, further comprising at least one of:
i) receiving input indicative of a bounding volume defining an extent of the anatomical region in the received historic volumetric image data; and
wherein the generating, using the neural network, the predicted subsequent volumetric image data, is constrained by generating the predicted subsequent volumetric image data only within the bounding volume: or
receiving input indicative of a bounding area defining an extent of the anatomical region in the
received subsequent projection image data; and
wherein the generating, using the neural network , the predicted subsequent volumetric image data, is constrained by generating the predicted subsequent volumetric image data for a volume corresponding to the bounding area in the received subsequent projection image data.

11. The computer-implemented method according to claim 16, wherein the neural network is trained to generate, from the volumetric image data representing the anatomical region at the first point in time, the predicted volumetric image data representing the anatomical region at the second point in time, by:
receiving volumetric training image data representing the anatomical region at an initial time step:
receiving two-dimensional training image data representing the anatomical region at a plurality of time steps in a sequence after the initial time step;
inputting, into the neural network, the received volumetric training image data for the initial time step; and
for one or more time steps in the sequence after the initial time step;
generating, with the neural network, predicted volumetric image data for the time step;
projecting the predicted volumetric image data for the time step, onto an image plane of the received two-dimensional training image data for the time step; and
adjusting the parameters of the neural network based on a first loss function representing a difference between the projected predicted volumetric image data for the time step, and the received two-dimensional training image data for the time step.

12. The computer-implemented method according to claim 11, wherein the neural network is trained to predict the volumetric image data representing the anatomical region at the second point in time, by further:
receiving volumetric training image data corresponding to the two-dimensional training image data (at one or more of the time steps in the sequence after the initial time step; and
wherein the adjusting is based further on a second loss function representing a difference between the predicted volumetric image data for the time step, and the received volumetric training image data for the time step.

13. The computer-implemented method according to claim 11, wherein the image plane of the received two-dimensional training image data for the time step is determined by i) registering the received two-dimensional training image data for the time step to the received volumetric training image data for the initial time step, or by ii) registering the received two-dimensional training image data for the time step to the predicted volumetric training image data for the time step.

14. The computer-implemented method according to claim 11; wherein the received volumetric training image data represents the anatomical region at an initial time step in a plurality of different subjects:
wherein the received two-dimensional training image data comprises a plurality of sequences, each sequence representing the anatomical region in a corresponding subject at a plurality of time steps in a sequence after the initial time step for the corresponding subject: and
wherein the inputting, the generating, the projecting, and the adjusting, are performed with the received volumetric training image data and the received two-dimensional training image data for each subject.

15. A non-transitory computer-readable storage medium having stored a computer program product comprising instructions which, when executed by one or more processors, cause the one or more processors to:
receive historic volumetric image data representing the anatomical region at a historic point in time;
receive subsequent projection image data representing the anatomical region at a subsequent point in time that is subsequent to the historical point in time; and
predict subsequent volumetric image data representing the anatomical region at the subsequent point in time based on the historic volumetric image data and the subsequent projection image data, wherein the prediction of the subsequent volumetric image data is constrained by the subsequent projection image data.

16. The computer-implemented method according to claim 1, further comprising:
inputting the received historic volumetric image data into a neural network; and
using the neural network, predicting the subsequent volumetric image data representing the anatomical region at the subsequent point in time,
wherein the neural network is trained to predict, from first volumetric image data representing the anatomical region at a first point in time and second projection image data representing the anatomical region at a second point in time subsequent to the first point in time, second volumetric image data representing the anatomical region at the second point in time, the prediction of the second volumetric image data is constrained by the second projection image data.

17. The non-transitory computer-readable storage medium according to claim 15, wherein the instructions, when executed by the one or more processors, further cause the one or more processors to:
input the received historic volumetric image data into a neural network: and
using the neural network, predict the subsequent volumetric image data representing the anatomical region at the subsequent point in time,
wherein the neural network is trained to predict, from first volumetric image data representing the anatomical region at a first point in time and second projection image data representing the anatomical region at a second point in time subsequent to the first point in time, second volumetric image data representing the anatomical region at the second point in time, the prediction of the second volumetric image data is constrained by the second projection image data.
